(19) United States
(12) Patent Application Publication
(10) Pub. No.: US 2022/0347373 A1
(43) Pub. Date: Nov. 3, 2022

(54) Extensible Conduit For The Communication Of Negative Pressure And Fluids

(71) Applicant: KCI Licensing, Inc., San Antonio, TX (US)

(72) Inventors: Christopher Brian LOCKE, Bournemouth (GB); Timothy Mark ROBINSON, Shillingstone (GB)

(21) Appl. No.: 17/633,519

(22) PCT Filed: Aug. 11, 2020
(86) PCT No.: PCT/IB2020/057542

§ 371 Date: Feb. 7, 2022

Related U.S. Application Data
(60) Provisional application No. 62/886,209, filed on Aug. 13, 2019.

(51) Int. Cl. A61M 1/00

(52) U.S. Cl. CPC A61M 1/86; A61M 1/85; A61M 2205/587; A61M 2205/0288; A61M 2205/3344

(57) ABSTRACT

A conduit includes a first end configured to be fluidly coupled to a wound site, and a second end configured to be fluidly coupled to a canister. The conduit additionally includes a central lumen extending between the first end and the second end. The conduit further includes a male interlocking member on a first side of the conduit and a female interlocking member on a second side of the conduit. Both the first side and the second side extend along a length of the conduit between the first end and the second end. The female interlocking member is configured to receive the male interlocking member.

### CROSS-REFERENCE TO RELATED APPLICATIONS

This application claims the benefit of priority to U.S. Provisional Application No. 62/886,209, filed on Aug. 13, 2019, which is incorporated herein by reference in its entirety.

### BACKGROUND

The present disclosure relates generally to the management of conduit used in medical devices. More specifically, the present disclosure relates to methods and systems for managing the length of conduit used in negative pressure wound therapy (NPWT) devices and instillation therapy (IT) devices.

NPWT and IT devices rely on conduit or flow tubing to transport and receive fluids from a wound site. These devices are often provided with a fixed length of conduit. The conduit must be long enough to provide mobility to a patient.

### SUMMARY

One implementation of the present disclosure is a conduit. The conduit includes a first end configured to be fluidly coupled to a wound site, and a second end configured to be fluidly coupled to a canister. The conduit additionally includes a central lumen extending between the first end and the second end. The conduit further includes a male interlocking member on a first side of the conduit and a female interlocking member on a second side of the conduit. The second side is disposed on an opposite side of the conduit as the first side. Both the first side and the second side extend along a length of the conduit between the first end and the second end. The female interlocking member is configured to receive the male interlocking member.

In some embodiments, the male interlocking member is configured to detachably couple to the female interlocking member when at least part of the conduit is in a coiled configuration. In some instances, the male interlocking member and the female interlocking member are detachably coupled to the conduit. In other instances, the male interlocking member and the female interlocking member are integrally formed with the conduit.

In any of the above embodiments, the male interlocking member and the female interlocking member may extend along an entire length of the conduit, from the first end to the second end. In some instances, a wall thickness of the first side of the conduit is greater than a wall thickness of the second side of the conduit. For example, the male interlocking member may include an extension piece extending from an outer surface of the conduit substantially normal to an axis of the conduit and the female interlocking member may include a recessed area extending inwardly from the outer surface.

In some embodiments, the male interlocking member includes a first magnetic material and the female interlocking member includes a second magnetic material of opposite polarity as the first magnetic material. Alternatively, or in combination, the male interlocking member and the female interlocking member may include a hook and loop fastener.

In any of the above embodiments, the conduit may have one of a circular cross-section or a rectangular cross-section. In some embodiments, the conduit is configured for use with a negative pressure wound therapy system in which the conduit further includes a plurality of outer lumens extending along the length of the conduit from the first end to the second end.

Another implementation of the present disclosure is a wound therapy system. The wound therapy system includes a dressing configured to cover a wound, and a canister configured to collect a fluid from the wound and a conduit. The conduit includes a first end configured to be fluidly coupled to the dressing, a second end configured to be fluidly coupled to the canister, and a central lumen extending between the first end and the second end. The conduit further includes a male interlocking member on a first side of the conduit and a female interlocking member on a second side of the conduit. The second side is disposed on an opposite side of the conduit as the first side. Both the first side and the second side extend along a length of the conduit between the first end and the second end. The female interlocking member is configured to receive the male interlocking member when at least part of the conduit is in a coiled configuration.

In some embodiments, the conduit is one of a plurality of conduits and the canister is one of a plurality of canisters. The conduits are configured to couple together using the male interlocking members and the female interlocking members to form a ribbon. In some instances, the system further includes a plurality of light sources each coupled to one of the canisters. Each one of the plurality of light sources may be configured to illuminate one of the canisters and at least a portion of the conduit connected to the canister. The plurality of light sources may be used to help identify different tubes. For example, each one of the plurality of light sources may be configured to produces a different color light. Alternatively, or in combination, each one of the plurality of conduits may be a different color.

In some embodiments, the conduit forms a single channel of a negative pressure wound therapy system or a wound instillation system.

Another embodiment of the present disclosure is a method of managing a fluid conduit for patient therapy. The method includes providing a conduit configured to interconnect between a wound therapy system and a dressing. The method additionally includes determining a desired length for the conduit. The method further includes engaging a plurality of interlocking members formed in a wall of the conduit to adjustably adapt a length of the conduit to the desired length.

In some embodiments, plurality of interconnecting members includes a male interconnecting member on a first side of the conduit and a female interlocking member on a second side of the conduit that is opposite the first side. The method of engaging the plurality of interlocking members may include positioning at least a portion of the conduit into a coiled configuration and pressing the male interlocking member into the female interlocking member along the portion of the conduit.

Another embodiment of the present disclosure is a method of initiating wound therapy. The method includes providing a conduit configured to interconnect between a wound therapy system and a dressing. The method additionally includes determining a desired length for the conduit. The method further includes engaging a plurality of interlocking members formed in a wall of the conduit to adjustably adapt a length of the conduit to the desired length. The method additionally includes providing a negative pressure to the dressing.

Another embodiment of the present disclosure is a wound therapy system. The wound therapy system includes a dressing, an NPWT system, an instillation system, and a plurality of conduits. The dressing is configured to cover a wound. The NPWT system is configured to collect a fluid from the wound. The instillation system is configured to provide an instillation fluid to a wound. The plurality of conduits includes a first conduit extending between the NPWT system and the dressing and a second conduit extending between the instillation system and the dressing. The plurality of conduits is coupled together along a length of the plurality of conduits to form a ribbon.

Another embodiment of the present disclosure is a wound therapy system. The wound therapy system includes a first dressing configured to cover a first wound, a second dressing configured to cover a second wound, an NPWT system, and a plurality of conduits. The NPWT system includes a first fluid canister configured to collect a fluid from the first wound and a second canister configured to collect a fluid from the second wound. The plurality of conduits includes a first conduit extending between the first fluid canister and the first dressing, and a second conduit extending between the second fluid canister and the second dressing. The plurality of conduits is coupled together along a length of the plurality of conduits to form a ribbon.

Another embodiment of the present disclosure is a conduit management member. The conduit management member includes a body, a male interlocking member, and a female interlocking member. The male interlocking member is disposed on a first side of the body that extends along a length of the body between a first end of the body and a second end of the body. The female interlocking member is disposed on a second side of the body opposite the first side. The female interlocking member is configured to receive the male interlocking member. The conduit management member is configured to detachably couple to a conduit of a wound therapy system.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is a perspective view of a conduit in a coiled configuration, according to an exemplary embodiment;

FIG. 2 is a side cross-sectional view of a portion of coiled configuration of FIG. 1;

FIG. 3 is a side cross-sectional view of the conduit of FIG. 1;

FIG. 4 is a side cross-sectional view of a conduit including interlocking members that are detachable from the conduit, according to an exemplary embodiment;

FIG. 5 is a perspective view of the interlocking members of FIG. 4;

FIG. 6 is a block diagram of a combined negative pressure wound therapy and instillation system including conduit in a coiled configuration, according to an exemplary embodiment;

FIG. 7 is a side view of a canister from a negative pressure wound therapy system, according to an exemplary embodiment;

FIG. 8 is a block diagram of a combined negative pressure wound therapy system including conduit in a coiled configuration, according to an exemplary embodiment;

FIG. 9 is a flow diagram of a method of managing a fluid conduit for a therapy system, according to an exemplary embodiment; and

FIG. 10 is a flow diagram of a method of engaging interlocking members on a fluid conduit for a wound therapy system, according to an exemplary embodiment.

### DETAILED DESCRIPTION

### Overview

Referring generally to the FIGURES, a conduit is provided that simplifies coiling and flat storage of excess conduit between a medical device and a patient or dressing. The conduit includes interlocking members that allow the conduit to be attached to itself (e.g., that allow one portion of the conduit to couple to another portion of the conduit) and/or to other conduits in a single unified ribbon. In various exemplary embodiments, the conduit includes a male interlocking member on a first side of the conduit and a female interlocking member on a second, opposing side of the conduit. The male interlocking member is configured to detachably (e.g., removably) couple to the female interlocking member so that the overall length of the conduit may be adjusted to suit different applications. The male and female interlocking members can be coextruded with the conduit as a single unitary structure which, advantageously, simplifies manufacturing of the conduit. Alternatively, at least one of the male or female interlocking members may be a separate piece from the conduit to form a conduit management system.

In various exemplary embodiments, the conduit forms part of a wound therapy system such as a NPWT system (configured to apply a negative pressure across a wound site) or an IT system (configured to supply instillation fluids to the wound site). In systems where multiple conduits are used, the interlocking members allow the different conduits to couple together. Among other benefits, coupling the various conduits together in a single “ribbon” allows a user to easily route multiple conduits from multiple systems along a single path and to keep track of individual conduits within the ribbon. In some embodiments, the therapy system additionally includes a plurality of light sources configured to illuminate at least one canister to which a conduit is connected. The light sources may be configured to each illuminate a different canister and at least a portion of the conduit connected to the canister. The light sources may be configured to activate automatically based on operating conditions of the therapy system (e.g., a flow condition through the conduit such as a blockage, etc.). Alternatively, or in combination, the light sources may each produce a different color light in order to help a patient or caregiver distinguish between the different conduits (e.g., to determine the function of each one of the conduits). These and other features and advantages of the conduit are described in detail below.

### Conduit Construction

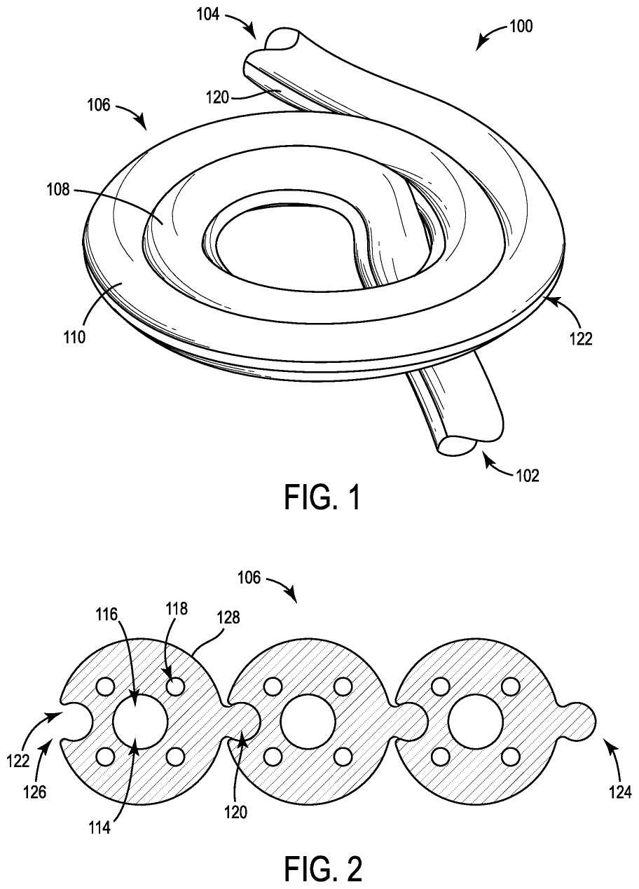

FIG. 1 provides a conduit 100, according to an exemplary embodiment. The conduit 100 may be a flow tube for a medical device. For example, the conduit 100 may be used with an NPWT system that is configured to apply a negative pressure to a wound site to promote healing of a wound. The conduit 100 may be configured to fluidly connect the NPWT system (e.g., canisters, pumps, etc.) to the wound site (e.g., to a medical dressing that covers the wound site, etc.). In some embodiments, the conduit 100 may alternatively or additionally be used with an IT system configured to supply instillation fluids (e.g., a topical wound treatment fluid or solution) to the wound site in order to facilitate healing of a wound and to prevent the wound from drying out. In yet other embodiments, the conduit 100 may be used with yet another medical system or device.

As shown in FIG. 1, the conduit 100 includes a first end 102 and a second end 104 that is opposite the first end 102. The first end 102 is configured to be fluidly coupled to a wound site. For example, the first end 102 may be configured to be fluidly coupled to a medical dressing (e.g., a drape, etc.) that is applied over the wound site. The second end 102 of the conduit 100 is configured to be fluidly coupled to a canister. The canister may be an air-tight fluid holding vessel or an open fluid reservoir. The canister may be configured to receive wound exudate and/or instillation fluids from the wound site.

The conduit 100 is reconfigurable between a coiled configuration and an uncoiled configuration. In the coiled configuration, the conduit 100 is at least partially coupled to itself. In other words, a first portion of the conduit 100 is coupled to a second portion of the conduit 100. In the coiled configuration shown in FIG. 1, the conduit 100 includes a plurality of coils 106 that are arranged with respect to one another in order to reduce the maximum distance between the medical device and the patient. A first, inner coil 108 of the plurality of coils 106 is coupled to a second, outer coil 110 of the plurality of coils 106 forming a generally flat spiral shape (e.g., a flattened coil, etc.) whose height is approximately the same as a diameter of each individual coil 108, 110. In other embodiments, the arrangement of the conduit 100 in the coiled configuration may be different. For example, multiple individual pieces of conduit 100 may be coupled together in a “ribbon” shape (e.g., a series of straight pieces of conduit 100 that are coupled together) that may be easily routed by a user to different areas within a medical environment. In the uncoiled configuration (not shown), the maximum allowable distance between the medical device and the patient is approximately equal to the overall length of the conduit 100 (e.g., the flow length through the conduit 100 between the first and second ends 102, 104).

As shown in FIG. 1, the conduit 100 includes interlocking members 120, 122 that allow the conduit 100 to detachably (e.g., removably) couple to itself along sides of adjacent coils 106. The interlocking members 120, 122 are shown to extend along an entire length of the conduit 100, from the first end 102 to the second end 104 which, advantageously, allows the coils 106 to be repositioned at any point along the length of the conduit 100 (e.g., at any position between the medical device and the patient).

Referring now to FIG. 2, a cross-section through three interconnected coils 106 is shown, according to an exemplary embodiment. The conduit 100 may include any number of flow channels 114 that fluidly couple the first end 102 to the second end 104 (e.g., that fluidly couple the medical device to the dressing). As shown in FIG. 2, the conduit 100 includes five flow channels 114 including a central lumen 116 and a plurality of outer lumens 118 surrounding the central lumen 116. In an NPWT system, the central lumen 116 may be configured to receive and transport fluids from the wound site (e.g., beneath the dressing) to the canister under a negative pressure. The outer lumens 118 may be configured to measure a condition within a space between the dressing and the wound site. For example, the outer lumens 118 may be configured to measure a negative pressure of the space by fluidly coupling the space to a pressure sensor.

As shown in FIG. 2, the interlocking members 120, 122 include a male interlocking member 120 and a female interlocking member 122. The male interlocking member 120 is disposed on a first side 124 of the conduit 100, which extends along a length of the conduit 100 between the first end 102 and the second end 104. The female interlocking member 122 is disposed on a second side 126 of the conduit 100. In various exemplary embodiments, the female interlocking member 122 is disposed on an opposite side of the conduit 100 as the male interlocking member 120. As shown in FIG. 2, the male interlocking member 120 and the female interlocking member 122 are separated approximately 180° along an outer perimeter 128 of the conduit 100. Among other benefits, positioning the interlocking members 120, 122 on opposite sides of the conduit 100 flattens the overall profile of the conduit 100 in the coiled configuration (see also FIG. 1), which can be preferable for storing the conduit 100 when not being used.

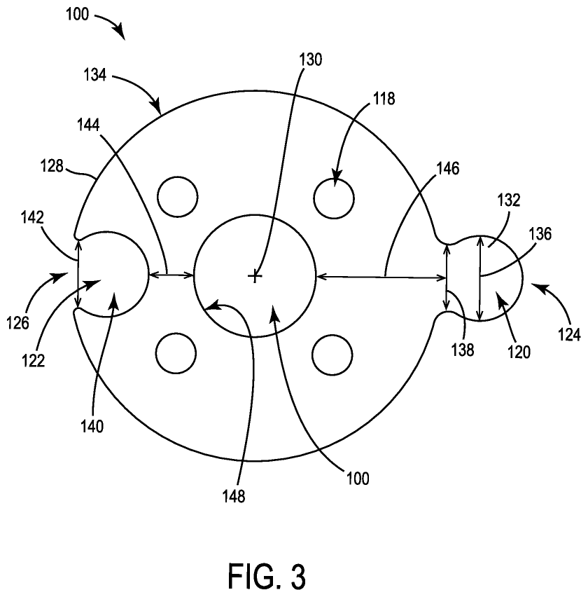

In the coiled configuration, as shown in FIG. 2, the male interlocking member 120 is detachably (e.g., removably) coupled to the female interlocking member 122 along a length of the conduit 100 between the first end 102 and the second end 104. FIG. 3 shows a cross-sectional view through a single section of conduit 100. The conduit 100 is shown to have a generally circular cross-section normal to a central axis 130 of the conduit 100 (e.g., relative to a flow direction of the conduit). As shown in FIG. 3, both the male interlocking member 120 and the female interlocking member 122 are formed with the conduit 100 as a single unitary structure. The conduit 100 may be extruded or otherwise formed from a plastic material such as plasticized polyvinylchloride (PVC) or another suitable medical grade material.

The interlocking members 120, 122 are configured to snap, clip, or otherwise detachably couple to one another in response to an applied force between the interlocking members 120, 122 (e.g., a force oriented substantially radially relative to the central axis 130 of the conduit 100). As shown in FIG. 3, the male interlocking member 120 includes an extension piece 132 extending outwardly from an outer surface 134 of the conduit 100 in a substantially perpendicular orientation relative to the central axis 130 of the conduit 100 (e.g., normal to the central axis 130, radially outward from the central axis 130, etc.). The extension piece 132 has a generally circular cross-sectional shape but may have other shapes in various exemplary embodiments. As shown in FIG. 3, a first width 136 of the extension piece 132 (e.g., a diameter of the extension piece 132) away from the outer surface 134 is greater than a second width 138 of the extension piece 132 proximate to the outer surface 134. The female interlocking member 122 includes a recessed area 140 (e.g., detent, slot, etc.) sized to receive and retain the male interlocking member 120 therein.

As shown in FIG. 3, the recessed area 140 extends inwardly from the outer surface 134 of the conduit 100 in a substantially perpendicular orientation relative to the central axis 130 (e.g., radially inward, etc.). The cross-sectional shape of the recessed area 140 is approximately the same as the male interlocking member 120 (e.g., generally circular in the embodiment shown in FIG. 3). A width of the female interlocking member 122, shown as third width 142 proximate to the outer surface 134 of the conduit 100 is less than the second width 138 of the male interlocking member 120 such that, during assembly, the female interlocking member 122 deforms to accommodate the male interlocking member 120.

The conduit 100 is structured to partially deform and flatten under compressive loading. As shown in FIG. 3, a wall thickness 144 of the first side 124 of the conduit 100, between the recessed area 140 and an inner surface 148 of the central lumen 116 (e.g., proximate to the female interlocking member 122, closer to the female interlocking member 122 than the male interlocking member 120) is greater than a wall thickness 146 of the second side 126 of the conduit 100 (e.g., proximate to the male interlocking member 120, closer to the male interlocking member 120 than the female interlocking member 122). Among other benefits, the smaller wall thickness 146 along the first side 124 allows the conduit 100 to at least partially collapse and thereby reduce the overall diameter of the conduit 100 under an applied pressure. The larger wall thickness 144 along the second side 126 of the conduit 100 prevents the conduit 100 from completely collapsing, which might otherwise restrict flow through at least one of the central and outer lumens 116, 118. The compliance of the conduit 100 under compressive loading can, advantageously, reduce the pressure a patient experiences when laying on top of the conduit 100.

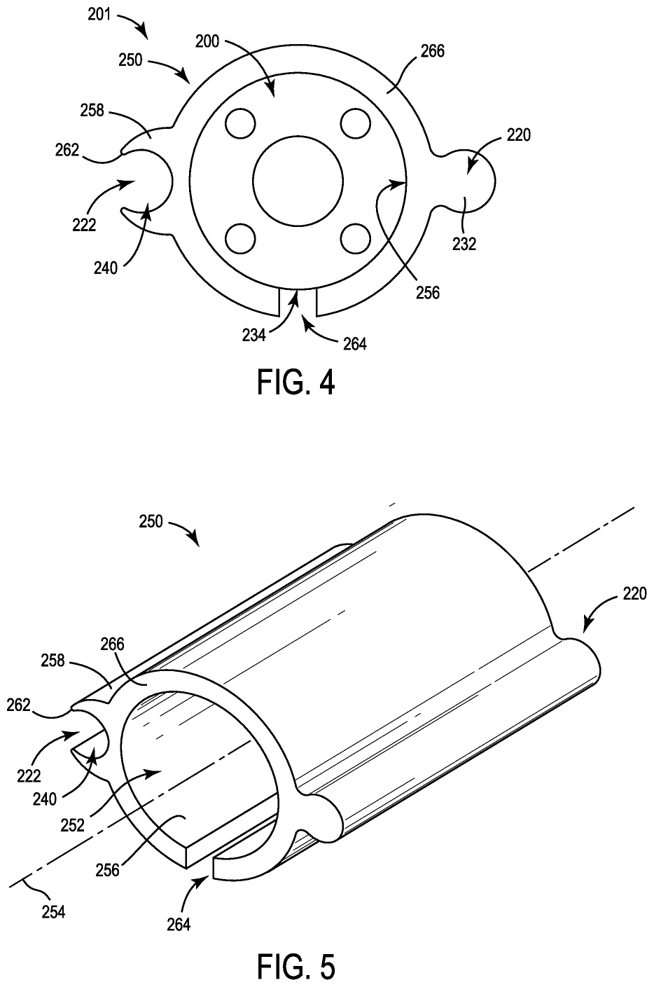

Referring now to FIGS. 4-5, a system 200 for conduit management is shown to include interlocking members 220, 222 that are detachably (e.g., removably) coupled to a conduit 201, according to an exemplary embodiment. The interlocking members 220, 222 include a male interlocking member 220 and a female interlocking member 222. The interlocking members 220, 222 are integrally formed with one another as a single unitary structure. The interlocking members 220, 222 form part of a conduit management member 250 that clips, hooks, snaps, latches, or otherwise fastens to the conduit 200. Similar to the conduit 201, the conduit management member 250 may be extruded or otherwise formed from a plastic material such as PVC or another suitable material. The length of the conduit management member 250 and/or the position of the conduit management member 250 along the conduit 201 may be modified depending on the holding force required to maintain the conduit 201 in the coiled configuration. According to various exemplary embodiments, the length of the conduit management member 250 is less than the overall length of the conduit 201.

As shown in FIG. 5, the conduit management member 250 includes an opening 252 extending through the conduit management member 250 along a central axis 254 of the conduit management member 250. The opening 252 is sized to receive the conduit 201 therein. As shown in FIG. 4, a cross-sectional shape of an inner surface 256 of the conduit management member 250 is approximately the same as a cross-sectional shape of an outer surface 234 of the conduit 201. In the exemplary embodiment of FIG. 4, the cross-sectional shape of the inner surface 256 is generally circular although other shapes are also envisioned (e.g., hexagonal, rectangular, etc.).

As shown in FIGS. 4-5, the size, shape, and position of the interlocking members 220, 222 is the same or similar to the interlocking members 220, 222 described with reference to FIGS. 1-3. However, unlike the female interlocking member 222 of FIGS. 1-3, the female interlocking member 222 of FIGS. 4-5 includes a second extension piece 258 extending outwardly from the conduit management member 250 in substantially perpendicular orientation relative to the central axis 254 of the conduit management member 250 (e.g., radially outward). The female interlocking member 222 also includes a recessed area 240 disposed in the second extension piece 258. The recessed area 240 may be the same or similar to the recessed area 140 of FIGS. 1-3.

A wall thickness of the female interlocking member 222 may be modified depending on the required holding force between the female interlocking member 222 and the male interlocking member 220. As shown in FIGS. 4-5, the wall thickness of the female interlocking member 222 gradually increases with decreasing radius from an outer edge 262 of the female interlocking member 222. Among other benefits, using a gradually increasing radius reduces the force required to couple the interlocking members 220, 222.

As shown in FIGS. 4-5, the conduit management member 250 includes an aperture 264 (e.g., slot, etc.) extending through an outer wall 266 of the conduit management member 250. In the embodiment of FIG. 5, the aperture 264 is generally rectangular. The aperture 264 may be resized by bending the conduit management member 250 (e.g., by pulling side walls of the aperture 264 apart) in order to clip or otherwise couple the conduit management member 250 onto the conduit 200. In other embodiments, another form of clip or fastener may be used to secure the conduit management member 250 in position over the conduit 200.

### Additional Configurations

The combination of features shown in the exemplary embodiments of FIGS. 1-3 and 4-5 should not be considered limiting. Many alternative implementations are possible without departing from the inventive concepts disclosed herein. For example, in some embodiments, the cross-sectional shape of the conduit 100, 200 may be different (e.g., rectangular, hexagonal, elliptical, etc.). The shape and arrangement of interlocking members 120, 122, 220, 222 may also be different. For example, in an embodiment where the cross-sectional shape of the conduit is rectangular (e.g., square), the conduit may be bent or otherwise formed in an alternating back and forth or “zig-zag” arrangement. In such an arrangement, an extended length of the conduit in an uncoiled configuration may be approximately double the extended length of the conduit in a fully coiled configuration.

In various exemplary embodiments, the interlocking members may be disposed on adjacent surfaces of the rectangular conduit such that the surfaces are coupled together. The interlocking members may include a hook-and-loop fastener, an adhesive product, or another suitable fastener. In various exemplary embodiments, the interlocking members may include a combination of multiple fasteners. For example, in the embodiment of FIGS. 1-3, the interlocking members 120, 122 may additionally include a magnetic material. The male interlocking member 120 may include a first magnetic material and the female interlocking member 122 may include a second magnetic material. The first magnetic material and the second magnetic material may be of opposite polarity in order to apply a magnetic force between the interlocking members 120, 122, thereby urging them together. The magnetic material may be coextruded or otherwise formed into the interlocking members 120, 122 as a single unitary structure.

In some embodiments, the conduit may be configured to retract into a coiled configuration automatically when disconnected from at least one of the medical device or the dressing (e.g., one or a combination of the medical device and the dressing). The conduit may be bent into a desired coiled configuration (e.g., “zig-zag,” spiral, etc.) by applying heat to the conduit or by modifying a wall thickness of the conduit during the forming process. In an embodiment where a magnetic material is used, the magnetic force may be enough to urge the conduit into a coiled configuration without an applied external force when disconnected from one of the medical device and the dressing.

### Wound Therapy System

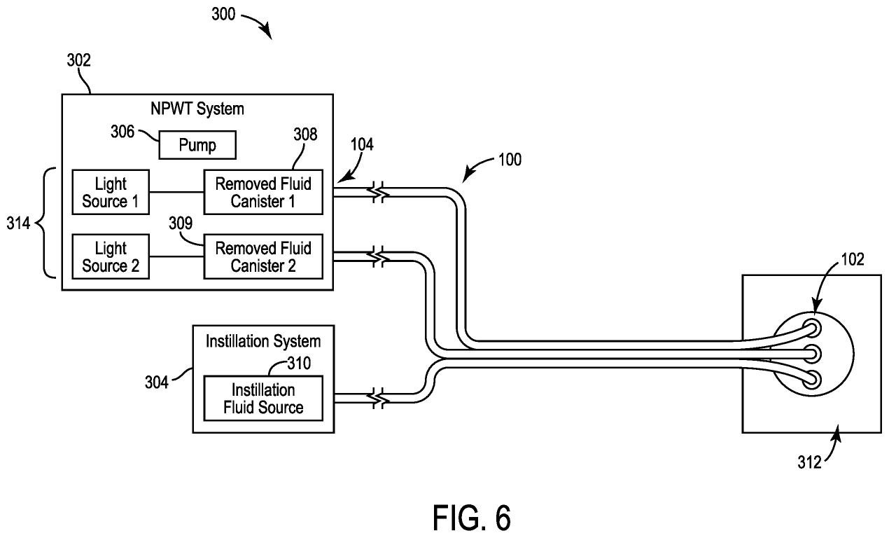

Referring now to FIG. 6, a wound therapy system 300 is shown to include both an NPWT system 302 and an IT system 304, according to an exemplary embodiment. In other embodiments, the wound therapy system 300 may include an NPWT system 302 in isolation from an IT system 304 or multiple NPWT systems. The NPWT system 302 includes a pump 306 and a plurality of canisters 308, 309. The pump 306 is configured to apply a negative pressure to a wound site and at least one of the canisters 308, 309 in order to collect wound exudate from the wound site and/or instillation fluid from the IT system 304. The NPWT system 302 additionally includes a plurality of conduits extending between the NPWT system 302 and a dressing 312 of the wound therapy system 300. The conduits fluidly couple the NPWT system 302 to the dressing 312. The conduits may be the same or similar to the conduits 100, 200 described with reference to FIGS. 1-3 and 4-5, respectively. For simplicity, similar numbering will be used to identify similar components.

As shown in FIG. 6, the IT system 304 includes an instillation fluid source 310 configured to provide (e.g., dispense) instillation fluid to a space between the dressing 312 and the wound site; for example, to prevent the wound site from drying out. The instillation fluid may also contain a medicant to help protect the wound site from infection. In one implementation, the NPWT system 302 may form at least part of a commercial NPWT system 302 such as the PREVENA™ Incision Management System and/or the V.A.C.RX4™ Therapy System by KCI. Similar to the NPWT system 302, the IT system 304 is fluidly coupled to the dressing 312 by a conduit, which may be the same or similar to the conduits 100 used for the NPWT system 302 (e.g., with male and female interlocking members 120, 122 but with only a single central lumen).

As shown in FIG. 6, a first end 102 of each of the plurality of conduits 100 is fluidly coupled to the dressing 312. A second end 104 of each of the plurality of conduits 100 is fluidly coupled to a corresponding one of the canisters 308, 309 or the instillation fluid source 310. The conduits 100 are shown in a coiled configuration in which the conduits 100 are coupled together in a “ribbon” extending at least a portion of a distance between the wound therapy system 300 and the dressing 312. In the embodiment of FIG. 6, the conduits 100 are detachably coupled to one another at approximately the same position between the NPWT and IT systems 302, 304 and the dressing 312. In other embodiments, and depending on the position of the IT system 304 with respect to the NPWT system 302, two or more conduits 100 may be coupled together at a plurality of different positions between the NPWT and IT systems 302, 304 and the dressing 312.

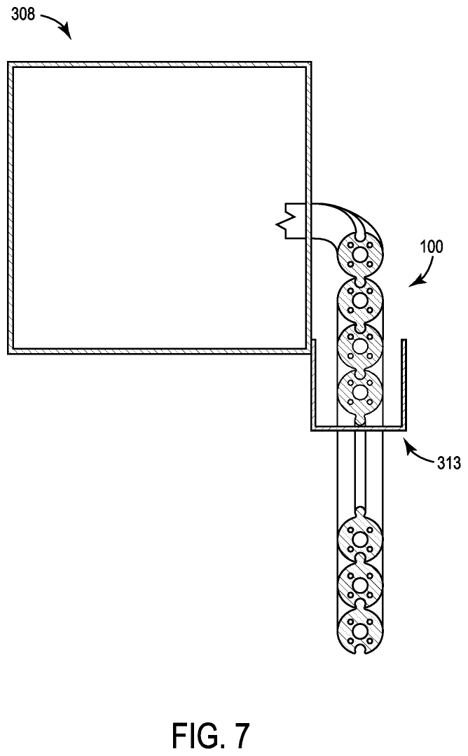

In some embodiments, a portion of at least one conduit 100 may be coupled to a corresponding one of the canisters 308, 309 in a coiled configuration. For example, FIG. 7 provides a canister 308 that is shown to include a retaining member 313. The retaining member 313 may be a hook, clip, strap, or another fastener configured to receive the conduit 100 in a coiled configuration. In the embodiment of FIG. 7, the retaining member 313 is a generally “U” shaped extension coupled to a lower edge of the canister 308. In one or more embodiments, the retaining member 313 may engage with at least one of the interlocking members 120, 122. For example, the retaining member 313 may include a female interlocking member configured to receive the male interlocking member 120 of the conduit 100 therein, and thereby detachably couple the canister 308 to the conduit 100 when the conduit is in a coiled configuration.

The wound therapy system 300 may further include features to help differentiate between conduits 100 used with different devices (e.g., the NPWT system 302 or the IT system 304) and/or between conduits 100 that are coupled to different canisters 308, 309 within a single device. In the exemplary embodiment of FIG. 6, the NPWT system 302 additionally includes a plurality of light sources 314. Each one of the light sources 314 is coupled to a corresponding one of the plurality of canisters 308, 309. In other embodiments, a single light source 314 is coupled to the plurality of canisters 308, 309. Each one of the light sources 314 may include a driver (e.g., power source) and an emitter. The emitter may be a light emitting diode, a fluorescent bulb or another type of light emitter. Each light source 314 may be coupled to a corresponding one of the canisters 308, 309 and may be configured to direct light into the canister 308, 309 through an opening in the canister (e.g., a window formed into an outer wall of the canister, etc.). Alternatively, each light source 314 may be disposed within a corresponding one of the canisters 308, 309.

In various exemplary embodiments, each light source 314 is configured to illuminate one of the canisters 308, 309 and at least a portion of the conduit 100 that is connected to the canister 308, 309. Among other benefits, the light sources 314 may be selectively activated to illuminate at least one of the conduits 100 in order to help a patient or caregiver identify the function of each conduit 100 (e.g., to identify which conduits are used for the NPWT system 302 as opposed to the IT system 304, etc.). For example, during instillation of each conduit 100 to the dressing 312, the caregiver may activate at least one of the light sources 314 via a user interface for the NPWT system 302 or the IT system 304. The caregiver may then connect the first end 102 of the illuminated conduit 100 to the corresponding port on a drape for the dressing 312. Among other benefits, using the light sources 314 to identify each conduit 100 during the instillation process simplifies assembly and makes it easier to diagnose issues in any one of the conduits 100. In some embodiments, each of the light sources 314 may be configured to produce a different color light in order to further differentiate between different conduits 100. Alternatively, in an implementation where a single light source 314 is used for multiple canisters 308, 309, filters may be provided to modify the color of light introduced to each canister 308, 309. In yet other embodiments, each one of the conduits 100 may be a different color (e.g., each of the conduits 100 may include a color pigment).

In some embodiments, the NPWT system 302 is configured to automatically activate at least one of the light sources 314 in response to an operating characteristic of the NPWT system 302. For example, the NPWT system 302 may be configured to activate the light source 314 in response to a blockage that is detected in one of the conduits 100. For example, the NPWT system 302 may include a pressure sensor configured to measure a pressure of the central lumen or an outer lumen. The pressure sensor may be communicatively coupled to a controller for the NPWT system 302. The controller may be configured to activate the light source 314 based on a determination that the measured pressure reported by the pressure sensor is greater than or less than a predefined threshold (e.g., a baseline pressure under nominal operating conditions, etc.). The light source 314 may illuminate the conduit 100 in which a blockage has been detected, thereby notifying the caregiver of the blockage and directing their attention to correct conduit 100.

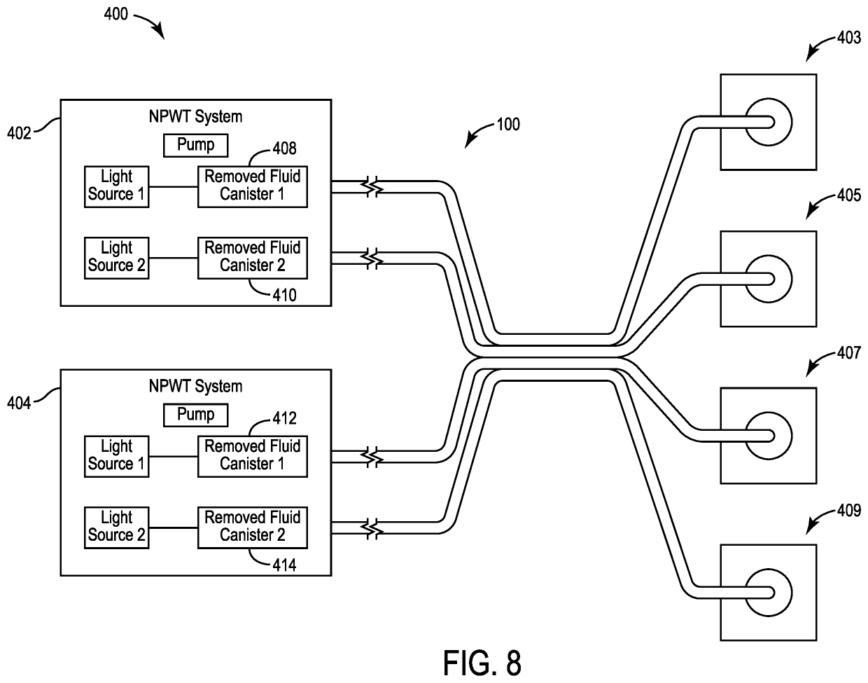

Referring now to FIG. 8, a wound therapy system 400 is shown to include a plurality of NPWT systems 402, 404 according to an exemplary embodiment. The NPWT systems 402, 404 are configured to treat multiple, discrete wound sites (e.g., individual dressings 403, 405, 407, 409 disposed on different areas of a patient's body, etc.). Each of the NPWT systems 402, 404 may be the same or similar to the NPWT system 302 of FIG. 6. As shown in FIG. 8, each NPWT system 402, 404 includes a plurality of conduits extending between the NPWT system 402, 404 and a corresponding one of the dressings 403, 405, 407, 409. Each one of the conduits fluidly couples a canister 408, 410, 412, 414 that is coupled to one of the NPWT systems 402, 404 to a corresponding one of the dressings 403, 405, 407, 409. The conduits may be the same or similar to the conduits, 100, 200 described with reference to FIGS. 1-3 and 4-5, respectively. For simplicity, similar numbering has been used to identify similar components. The conduits 100 are shown in a coiled configuration in which the conduits 100 are coupled together in a “ribbon” extending at least a portion of a distance between the NPWT systems 402, 404 and the dressings 403, 405, 407, 409. The number, type, and arrangement of NPWT systems 402, 404 may be different in various exemplary embodiments.

### Method of Managing a Fluid Conduit for Patient Therapy

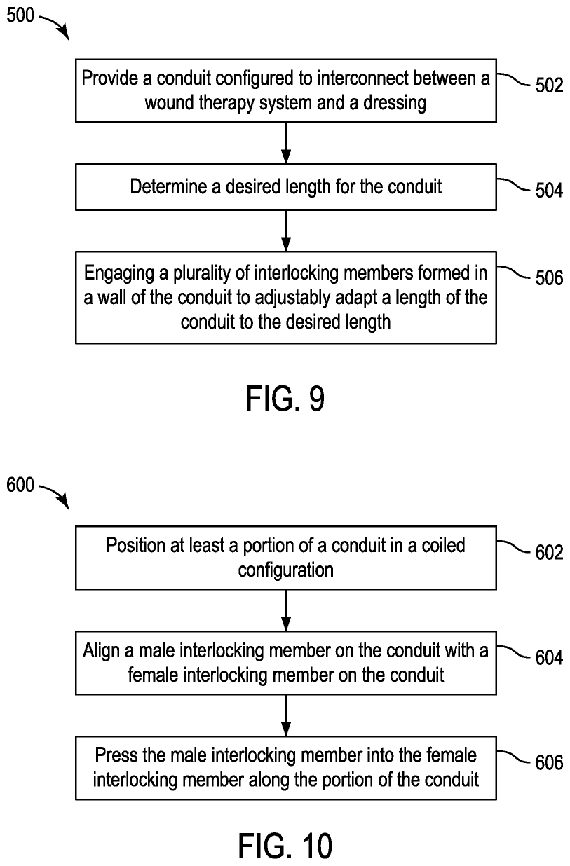

Referring to FIG. 9, a flow diagram of a method 500 of managing a fluid conduit for patient therapy is provided, according to an exemplary embodiment. The method 500 may be used to adapt a length of a single conduit or a plurality of conduits. At 502, a conduit 100 configured to interconnect between a wound therapy system and a dressing is provided. The fluid conduit may be the same or similar to the conduit 100 of FIGS. 1-3 (or, alternatively, the conduit 200 of FIG. 4). For convenience, similar number will be used to identify similar components. Block 502 may additionally include connecting the conduit 100 to the wound therapy system and/or the dressing. At 504, a desired length for the conduit is determined. Block 504 may include measuring a smallest distance between the dressing and the wound therapy system and/or coiling at least a portion of the conduit 100 to take up any excess length of conduit 100 between the wound therapy system and the dressing. In some embodiments, block 504 may include positioning the portion of the conduit 100 into a coiled configuration.

At 506, a plurality of interlocking members (e.g., male interlocking member 220 and female interlocking member 222) formed into a wall of the conduit 100 are engaged (e.g., coupled) to adjustably adapt a length of the conduit 100 to the desired length. FIG. 10 is a flow diagram of a method 600 of engaging the interlocking members on the conduit 100. At 602, at least a portion of the conduit 100 is positioned in a coiled configuration. Block 602 may include the various operations described with reference to block 404 of FIG. 9. At 604, the male interlocking member 220 of the conduit 100 is brought into alignment with the female interlocking member 222. Block 604 may include positioning the extension piece 132 of the male interlocking member 220 over the recessed area 140 of the female interlocking member 222. At 606, the male interlocking member 220 is pressed into the female interlocking member 222 along the portion of the conduit 100 to couple the male interlocking member 220 into the female interlocking member 222. In other embodiments, the methods 500, 600 of FIGS. 9-10, respectively, may include additional, fewer, and/or different operations.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

1. A conduit, comprising:
a first end configured to be fluidly coupled to a wound site;
a second end configured to be fluidly coupled to a canister;
a central lumen extending between the first end and the second end;
a male interlocking member on a first side of the conduit, wherein the first side extends along a length of the conduit between the first end and the second end; and
a female interlocking member on a second side of the conduit, wherein the second side extends along the length of the conduit, wherein the second side is opposite the first side, and wherein the female interlocking member is configured to receive the male interlocking member.

2. The conduit of claim 1, wherein the male interlocking member is configured to detachably couple to the female interlocking member when at least part of the conduit is in a coiled configuration.

3. The conduit of claim 1, wherein the male interlocking member and the female interlocking member extend along an entire length of the conduit, from the first end to the second end.

4. The conduit of claim 1, wherein the male interlocking member and the female interlocking member are integrally formed with the conduit.

5. The conduit of claim 1, wherein a wall thickness of the first side of the conduit is greater than a wall thickness of the second side of the conduit.

6. The conduit of claim 5, wherein the male interlocking member comprises an extension piece extending from an outer surface of the conduit substantially normal to an axis of the conduit, and wherein the female interlocking member comprises a recessed area extending inwardly from the outer surface.

7. The conduit of claim 1, wherein the male interlocking member comprises a first magnetic material, and wherein the female interlocking member comprises a second magnetic material of opposite polarity as the first magnetic material.

8. (canceled)

9. The conduit of claim 1, wherein the conduit is configured for use with a negative pressure wound therapy system, wherein the conduit further comprises a plurality of outer lumens extending along the length of the conduit from the first end to the second end.

10. The conduit of claim 9, wherein the outer lumens are configured to fluidly couple to pressure sensors and to a dressing to provide pressure feedback to the pressure sensors.

11. (canceled)

12. The conduit of claim 9, wherein each of the outer lumens is disposed between the central lumen and an outer surface of the conduit such that the outer lumens surround the central lumen.

13. The conduit of claim 1, wherein the central lumen is configured to fluidly couple to a negative pressure source and a dressing.

14. The conduit of claim 1, wherein the central lumen is configured to provide a negative pressure.

15. A wound therapy system, comprising:
a dressing configured to cover a wound;
a canister configured to collect a fluid from the wound;
a conduit, comprising:
a first end configured to be fluidly coupled to the dressing;
a second end configured to be fluidly coupled to the canister;
a central lumen extending between the first end and the second end;
a male interlocking member on a first side of the conduit, wherein the first side extends along a length of the conduit between the first end and the second end; and
a female interlocking member on a second side of the conduit, wherein the second side extends along the length the conduit, wherein the second side is opposite the first side, and wherein the female interlocking member is configured to receive the male interlocking member when at least part of the conduit is in a coiled configuration.

16. The system of claim 15, wherein the male interlocking member of the conduit is configured to detachably couple to the female interlocking member of the conduit.

17.-23. (canceled)

24. The system of claim 15, wherein the canister comprises a retaining member configured to support a section of coiled conduit.

25. The system of claim 15, wherein the conduit is one of a plurality of conduits and the canister is one of a plurality of canisters, wherein the conduits are configured to couple together using the male interlocking members and the female interlocking members to form a ribbon.

26. The system of claim 25, further comprising a plurality of light sources, wherein each one of the plurality of light sources is coupled to one of the canisters, and wherein each one of the plurality of light sources is configured to illuminate one of the canisters and at least a portion of the conduit connected to the canister.

27. The system of claim 26, wherein each one of the plurality of light sources produces a different color light.

28. The system of claim 27, wherein each one of the plurality of light sources is configured to illuminate in response to a blockage in a corresponding one of the conduits.

29. (canceled)

30. The system of claim 15, wherein the conduit comprises a single channel of a negative pressure wound therapy system or a wound instillation system.

31.-36. (canceled)

37. A method of managing a fluid conduit for patient therapy, comprising:
providing a conduit configured to interconnect between a wound therapy system and a dressing, the dressing configured to cover a wound;
determining a desired length for the conduit; and
engaging a plurality of interlocking members formed in a wall of the conduit to adjustably adapt a length of the conduit to the desired length.

38.-39. (canceled)

40. A method of initiating wound therapy, comprising:
providing a conduit configured to interconnect between a wound therapy system and a dressing, the dressing configured to cover a wound;
determining a desired length for the conduit;
engaging a plurality of interlocking members formed in a wall of the conduit to adjustably adapt a length of the conduit to the desired length; and
providing a negative pressure to the dressing.

41.-45. (canceled)

46. A wound therapy system, comprising:
a dressing configured to cover a wound;
an NPWT system configured to collect a fluid from a wound;
an instillation system configured to provide an instillation fluid to a wound; and
a plurality of conduits, a first conduit extending between the NPWT system and dressing, and a second conduit extending between the instillation system and the dressing, the plurality of conduits coupled together along a length of the plurality of conduits to form a ribbon.

47.-54. (canceled)

55. A wound therapy system, comprising:
a first dressing configured to cover a first wound;
a second dressing configured to cover a second wound;
an NPWT system, comprising:
a first fluid canister configured to collect a fluid from the first wound; and
a second fluid canister configured to collect a fluid from the second wound; and
a plurality of conduits, a first conduit extending between the first fluid canister and the first dressing, and a second conduit extending between the second fluid canister and the second dressing, the plurality of conduits coupled together along a length of the plurality of conduits to form a ribbon.

56.-63. (canceled)

64. A conduit management member, comprising:
a body;
a male interlocking member on a first side of the body, the first side extending along a length of the body between a first end of the body and a second end of the body; and
a female interlocking member on a second side of the body, the second side extending along a length of the body that is opposite the first side, the female interlocking member configured to receive the male interlocking member, the conduit management member configured to detachably couple to a conduit of a wound therapy system.

65.-68. (canceled)
